(19) United States
(12) Patent Application Publication
(10) Pub. No.: US 2024/0032515 A1
(43) Pub. Date: Feb. 1, 2024

(54) METHOD FOR PRODUCING BIRD PRODUCING HUMAN IMMUNOGLOBULIN FC AND TARGET PROTEIN

(71) Applicant: AVINNOGEN CO., LTD., Seoul (KR)

(72) Inventors: Jae Yong HAN, Seoul (KR); Jin Se PARK, Yongin-si (KR)

(73) Assignee: AVINNOGEN CO., LTD., Seoul (KR)

(21) Appl. No.: 18/473,448

(22) Filed: Sep. 25, 2023

(30) Foreign Application Priority Data
Apr. 1, 2021 (KR) 10-2021-0042615

(51) Int. Cl. A01K 67/027; C12N 15/11; C12N 9/22; C07K 14/76

(52) U.S. Cl. CPC A01K 67/0278; C12N 15/11; C12N 9/22; C07K 14/76; C12N 2310/20

(57) ABSTRACT

The present invention relates to a bird in which human immunoglobulin G (hIgG) Fc, a protein fused with a human-derived protein and Fc, or a monoclonal antibody is expressed in a hepatocyte-specific manner, and a method for producing the same. Birds produced by the production method of the present invention can economically mass-produce human immunoglobulin Fc with enhanced anti-inflammatory effects in egg yolk and blood.

### TECHNICAL FIELD

The present invention relates to a bird capable of mass-producing human IgG Fc and Fc-fusion proteins with enhanced anti-inflammatory effects, and a method for producing the same.

### BACKGROUND ART

An autoimmune disease (immune thrombocytopenia, Guillain-Barre syndrome, lupus, chronic inflammatory demyelinating polyneuropathy, and the like) is a type of immune disease caused by the activation of abnormal inflammatory responses due to abnormalities in the immune system and the production of antibodies against self-antigens. In order to treat such autoimmune diseases, a method of administering polyclonal IgG isolated and purified from human blood as an injection (intravenous immunoglobulin, IVIG) has been adopted. Since the dose of therapeutic IVIG for suppressing inflammatory responses and immune activity is at the level of 0.4 to 2 g/kg and thus very high concentrations of IgG are required, blood collected from thousands to tens of thousands of donors is required. However, the amount of donated blood is very insufficient compared to an increase in the prevalence of inflammatory and autoimmune diseases. Although there has been an attempt to produce IVIG by an animal cell culture method in order to solve the above problems, there is a limitation in producing IVIG by a recombinant method because there is a cost problem due to high-concentration administration and non-human glycans produced by animal cells may cause allergic reactions upon administration at high concentrations. Therefore, there is a need for developing a material capable of being mass-produced and economically produced in place of human blood in order to efficiently produce IVIG. Since chickens do not produce non-human glycans, biopharmaceuticals produced from chickens are less likely to cause allergic reactions in humans. Further, the liver continuously produces plasma proteins, and such proteins are characterized by large accumulations in egg yolk. Therefore, when biopharmaceuticals can be produced in a hepatocyte-specific manner, it is possible to mass-produce proteins required in egg yolk and blood.

A chicken is an animal with a high egg production rate, and has been considered an effective foreign protein production system because it produces 300 or more eggs per year and the albumen and yolk of eggs have a high protein content. Since proteins formed in chicken hepatocytes are secreted into the blood or accumulated in egg yolk at high levels, foreign proteins are easily mass-produced and isolated using these characteristics.

In the case of a chicken liver, since the expression and activity of α-2,6 sialyltransferase (ST6GAL1), which forms the sialic acid glycan structure of proteins, are high, proteins produced in hepatocytes are characterized by α-2,6 sialylation with high efficiency. In the case of Fc with an improved α-2,6 sialylation rate, it is possible to efficiently improve the expression of FcγRIIB, which acts on immune cells to suppress immune activity. In addition, glycan sialylation plays an important role in maintaining the in vivo half-life of protein pharmaceuticals. Therefore, in the case of protein pharmaceuticals produced from a chicken liver, the efficient sialylation of glycans may prevent the problem of a reduction in in vivo activity of biopharmaceuticals due to a reduction in half-life.

Another characteristic of chicken hepatocytes is the afucosylation of proteins due to the low activity of α-1,6 fucosyltransferase (FUT8), which is a major fucosylation-causing enzyme. Non-fucosylated Fc may bind strongly to FcγRIIIA to saturate FcγRIIIA more efficiently, as well as more efficiently achieve ITAMi signaling through FcγRIIIA According to the characteristics described above, when human IgG Fc and Fc-bound fusion proteins (hIgG Fc and Fc fusion proteins) are produced using the chicken liver, the sialylation rate is improved, and non-fucosylated Fc can be mass-produced.

Similar to a chicken liver, the human liver also has the characteristics of high ST6GAL1 activity and low FUT8 activity. Therefore, proteins produced in the human liver and secreted into the blood have the characteristics of a high α-2,6 sialylation rate and a low fucosylation rate, which are the same as the protein produced and secreted in/from the chicken liver. In addition, since chickens do not form non-human glycans, glycan forms that are not found in humans are not found in proteins produced in the chicken liver. Therefore, the N-glycan patterns of proteins produced in the chicken liver and the human liver have the same characteristics. Therefore, when factors such as Factor VIII, Factor IX, and alpha-1-antitrypsin, which are originally produced in the human liver, are produced in the chicken liver, factors with the same N-glycan pattern as humans may be efficiently produced, which makes it possible to prevent the suppression of antibody formation and a reduction in in vivo activity due to differences in N-glycan patterns.

In addition, the fucosylation deficiency of Fc may induce antibody-dependent cell cytotoxicity (ADCC) to greatly increase the in vivo activity of anti-CD20 monoclonal antibodies, anti-HER2 monoclonal antibodies, anti-EGFR monoclonal antibodies, and the like, which are types of anti-cancer antibodies that attack cancer cells. Furthermore, even in the case of an anti-TNF-alpha monoclonal antibody, which is used as a therapeutic agent for autoimmune diseases, the defucosylation of the Fc site is known to improve anti-inflammatory responses by effectively suppressing the activity of T cells. Therefore, when therapeutic monoclonal antibodies described above are produced in the chicken liver, monoclonal antibodies with dramatically improved in vivo activity may be economically extracted from egg yolks and blood in large amounts.

Due to such characteristics, when hIgG Fc, Fc fusion proteins and monoclonal antibodies are produced in the chicken liver, not only therapeutic agents with enhanced anti-inflammatory effects compared to existing human blood-derived IVIG, but also various Fc fusion proteins consisting of proteins with the same pattern as human N-glycans, and monoclonal antibodies with increased in vivo activity can be accumulated in large amounts in egg yolk and blood, and can be economically mass-produced.

### DISCLOSURE

### Technical Problem

An object of the present invention is to provide a method for producing a genome-edited bird that produces human immunoglobulin G (hIgG) Fc, Fc fusion proteins and monoclonal antibodies with enhanced anti-inflammatory effects using a vector into which a hepatocyte-specific expressed gene and a gene encoding hIgG Fc or an Fc fusion protein are introduced.

### Technical Solution

1. A vector into which a hepatocyte-specific expressed gene, a gene encoding an Fc region of an antibody, and a gene encoding a target protein are introduced.
2. The vector of 1 above, in which the hepatocyte-specific expressed gene is at least one selected from the group consisting of albumin (alpha-livetin), beta-livetin, vitellogenin, apovitellenin, alpha-2-macroglobulin, apolipoprotein, transferrin and fibrinogen.
3. The vector of 1 above, in which the target protein is at least one selected from the group consisting of human immunoglobulin G (hIgG), factor VIII, factor IX, alpha-1-antitrypsin, erythropoietin, growth hormone, colony-stimulating factor, interferon, insulin and glucagon-like peptide-1, a CD20 antibody, a HER2 antibody, an EGFR antibody and a TNF-alpha antibody.
4. The vector of 1 above, in which the hepatocyte-specific expressed gene does not include a stop codon thereof.
5. The vector of 1 above, further including a gene encoding a 2A peptide between the hepatocyte-specific expressed gene and the gene encoding human immunoglobulin G (hIgG) Fc.
6. The vector of 5 above, in which the 2A peptide is at least one selected from the group consisting of T2A, P2A, F2A and E2A.
7. The vector of 5 above, further including a gene encoding a secretory signal peptide downstream of the gene encoding a 2A peptide.
8. A composition for producing a target protein including the vector of any one of 1 to 7 above.
9. A bird expressing a gene encoding a target protein in hepatocytes.
10. The bird of 9 above, in which the bird is produced by a method including: transducing avian germ cells with the vector of any one of 1 to 7 above; and obtaining a germline chimeric bird by transplanting the transduced germ cells into avian embryos.
11. The bird of 10 above, in which the bird is produced by the method further including: obtaining a heterozygous progeny generation by crossing the germline chimeric bird with a wild-type bird; and obtaining a homozygous progeny generation by crossing the heterozygous progeny generation.
12. The bird of 9 above, in which the bird is a chicken, a quail, a pheasant, a turkey, or a duck.
13. A method for producing a bird, the method including: transducing avian germ cells with the vector of any one of 1 to 7 above; and obtaining a germline chimeric bird by transplanting the transduced germ cells into avian embryos.
14. The method of 13 above, further including: obtaining a heterozygous progeny generation by crossing the germline chimeric bird with a wild-type bird; and obtaining a homozygous progeny generation by crossing the heterozygous progeny generation.
15. The method of 13 above, in which the bird is a chicken, a quail, a pheasant, a turkey, or a duck.
16. A method for producing a target protein, the method including purifying a target protein in an egg yolk or blood of the bird of any one of 9 to 12 above.
17. A target protein obtained from an egg yolk or blood of the bird of any one of 9 to 12 above.

### Advantageous Effects

The vector into which the hepatocyte-specific expressed gene and the gene encoding the human immunoglobulin G (hIgG) Fc of the present invention are introduced and the method for producing a bird expressing hIgG Fc in hepatocytes can economically mass-produce human immunoglobulin Fc with enhanced anti-inflammatory effects, various Fc-fusion proteins, and therapeutic monoclonal antibodies in the egg yolk and blood of a chicken.

### DESCRIPTION OF DRAWINGS

FIG. 1 relates to the structure of a vector for producing an ALB-hIgG tagged genome-edited chicken, (A) the structure of a donor plasmid and an sgRNA-Cas9 plasmid. The donor plasmid includes intron 13 of ALB, exon 14 of ALB, a T2A coding sequence, an ALB signal peptide coding sequence, an hIgG Fc coding sequence and an ALB 3′ UTR. (B) After translation, hIgG Fc is linked to ALB by the T2A peptide, and the T2A peptide is cleaved in the cytoplasm to generate two separate proteins ALB and hIgG Fc.

FIG. 2 shows the establishment of ALB-hIgG Fc-tagged primordial germ cells (PGCs). (A) To express hIgG Fc in a liver-specific manner, ALB was tagged with the hIgG Fc coding sequence. A donor vector including ALB intron 13, ALB exon 14 without a stop codon, a T2A peptide coding sequence, an ALB signal peptide coding sequence, an hIgG Fc coding sequence, an ALB 3 'UTR and a puromycin resistance gene was constructed. When PGCs were co-transduced with a donor vector targeting ALB intron 13 and a single guide RNA (sgRNA), the donor vector was inserted into an sgRNA target site. As a result, a modified allele has ALB exon 14 without a stop codon, a T2A coding sequence, an ALB signal peptide coding sequence and an hIgG Fc coding sequence. (B) Knock-in verification of PGCs using a specific primer for intron 13 of ALB and hIgG Fc. (C) Sequencing analysis of TA-cloned PCR products of knock-in PGCs.

FIG. 3 relates to the flow of an ALB-hIgG Fc-tagged chicken production experiment, in which a tagging plasma and an sgRNA-Cas9 plasmid are introduced into cultured chicken PGCs. After transduction, ALB-hIgG Fc-tagged genome-edited PGCs were selected by puromycin treatment. Selected genome-edited PGCs were transplanted into recipient Korean Ogye embryos (i/i) and mated with wild-type white leghorn hens (I/I) to produce donor PGC-derived chickens (I/I). Among donor PGC-derived chickens, ALB-hIgG Fc Tagged genome-edited chickens were confirmed using PCR and Sanger sequencing.

FIG. 4 relates to the production of ALB-hIgG Fc tagged genome-edited chickens, (A) production of donor PGC-derived progeny by a test cross between germline chimeras (i/i) and wild-type hens (I/I). (B) Genomic DNA analysis of donor PGC-derived progeny by PCR and Sanger sequencing. Red letters indicate the PAM sequence and blue letters indicate the sgRNA target site.

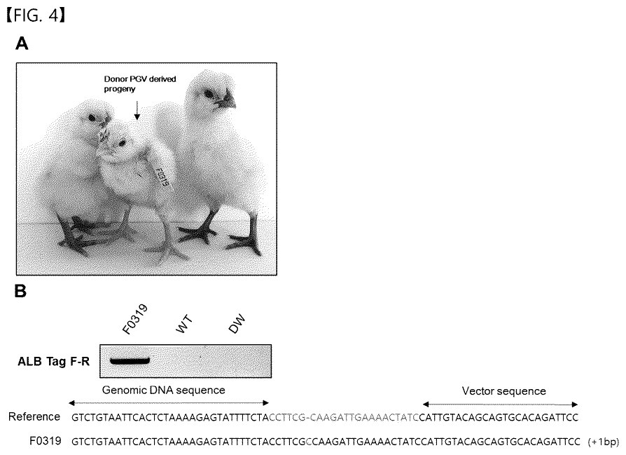

FIG. 5 relates to hIgG Fc production in serum from ALB-hIgG Fc-tagged genome-edited chickens, (A) Western blot of ALB-hIgG Fc-tagged genome-edited chicken serum using an anti-human IgG antibody (wild-type white leghorn serum was used as a control). (B) SDS-PAGE and Coomassie blue staining of ALB-hIgG Fc-tagged genome-edited chicken serum. Serum was diluted 10-, 50-, and 100-fold in DW (wild-type white leghorn serum was used as a control). (C) Concentration of hIgG Fc estimated by ELISA in ALB-hIgG Fc-tagged genome-edited chicken serum at week 15 and week 20.

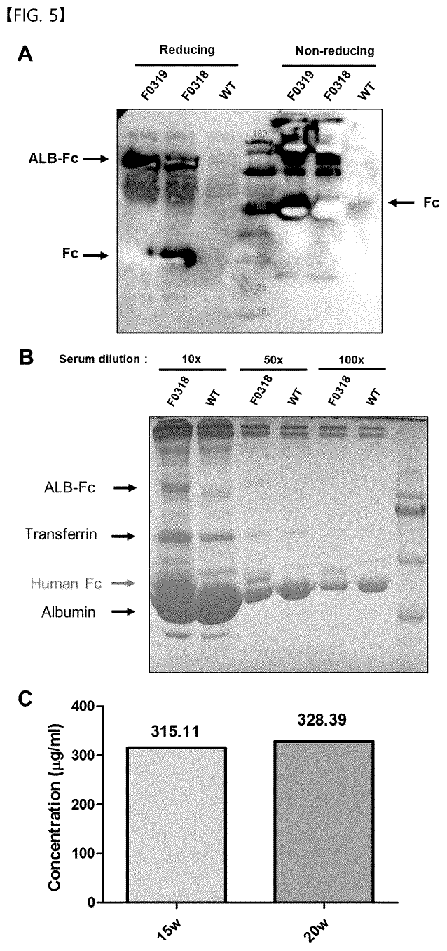

FIG. 6 relates to the N-glycosylation pattern profiling of chicken liver-derived hIgG Fc, (A) hIgG Fc was purified from ALB-hIgG Fc genome-edited chicken serum using a Protein A column and size exclusion chromatography, and purified hIgG Fc N-glycan profiles were analyzed by UPLC/MS. (B) Percentage of total intensity for each N-glycan. Red boxes indicate N-glycans including sialic acid.

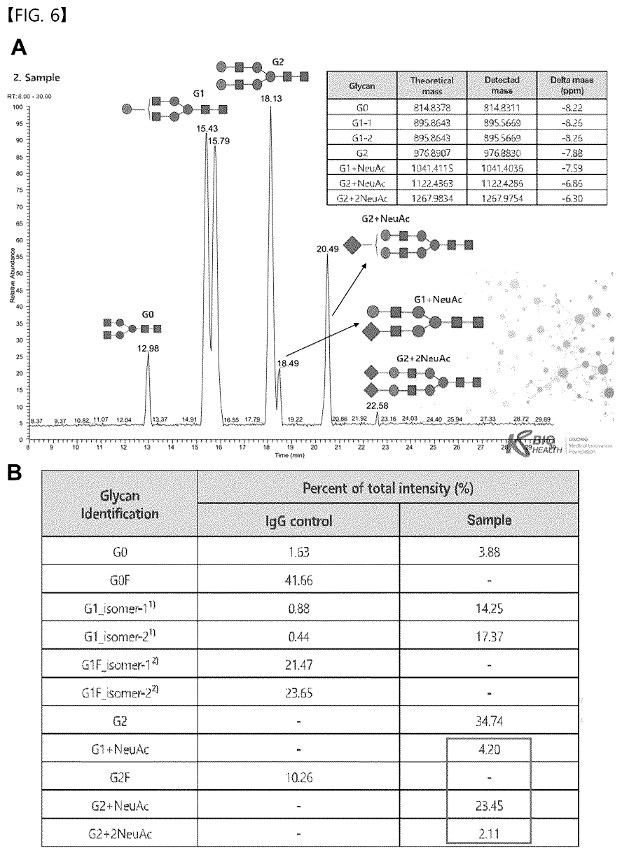

### MODES OF THE INVENTION

Hereinafter, the present invention will be described in detail.

The present invention may provide a vector into which a hepatocyte-specific expressed gene, a gene encoding an Fc region of an antibody, and a gene encoding a target protein are introduced.

“Hepatocyte-specific expressed gene” is a gene that is specifically expressed in hepatocytes through interactions with a liver-enriched transcription factor (LEF) at the transcriptional stage, and may express foreign genes in a tissue-specific manner.

A hepatocyte-specific expressed gene encodes a hepatocyte-specific expressed protein, and the hepatocyte-specific expressed gene/protein may be, for example, albumin (alpha-livetin), beta-livetin, vitellogenin, apovitellenin, alpha-2-macroglobulin, apolipoprotein, transferrin or fibrinogen.

The liver continuously produces plasma proteins, some of which have the characteristics of being accumulated in a large amount in egg yolk. Therefore, when foreign genes are specifically expressed in liver tissue, proteins encoded by the foreign genes are continuously secreted into the plasma, and in this case, the proteins encoded by the foreign genes can be accumulated in a large amount in egg yolk as long as they are known to be capable of being accumulated in egg yolk. The crystallization fragment (Fc) region is a fragment crystallizable region of an antibody, and is known to interact with cell surface receptors called Fc receptors and some proteins of the complement system to activate the immune system, and can serve to allow the produced target protein to be accumulated in egg yolk.

The antibody is used synonymously with immunoglobulin and may be, for example, IgG, IgA, IgM, IgD or IgE, preferably IgG, but is not limited thereto.

Immunoglobulin (IgG) is one of the individual types of antibodies which are monomers consisting of two identical heavy chains and a light chain, is most abundant in the blood stream, and accounts for 75% of the total amount of immunoglobulins present in serum in the case of humans.

The target protein is a protein to be overexpressed and mass-produced, and may be any protein that can be used as a biopharmaceutical, such as human immunoglobulin G (hIgG), factor VIII, factor IX, alpha-1-antitrypsin, erythropoietin, growth hormone, colony stimulating factor, interferon, insulin and glucagon-like peptide-1, a CD20 antibody, a HER2 antibody, an EGFR antibody or a TNF-alpha antibody, but is not limited thereto.

The vector is a means for expressing a specific gene, can be replicated in cells, may allow a gene to be expressed, and may be a concept including a plasmid, a bacterial artificial chromosome (BAC), a yeast artificial chromosome (YAC), a retrovirus, an adenovirus, and a non-viral vector but is not limited thereto.

A hepatocyte-specific expressed gene may not include a stop codon thereof.

The hepatocyte-specific expressed gene may not include a stop codon because a downstream gene encoding a target protein should be expressed.

The vector of the present invention may further include a gene encoding a 2A peptide between a hepatocyte-specific expressed gene and a gene encoding a target protein.

The 2A peptide consists of 18 to 22 amino acid sequences, and has a characteristic that the amino acid sequence is cleaved (self-cleavage) by inducing ribosome skipping during protein translation in cells, and thus may serve to ensure that two types of proteins are expressed almost equally.

The 2A peptide is located between the hepatocyte-specific expressed gene and the target protein-encoding gene and is cleaved within the cell to allow the hepatocyte-specific expressed gene and the target protein portion to be expressed, respectively, and may be located at the coding sequence (CDS) 3′ end of the hepatocyte-specific expressed gene. The type of 2A peptide may be, for example, T2A, P2A, F2A or E2A, but is not limited thereto.

The vector of the present invention may further include a gene encoding a secretory signal peptide downstream of the gene encoding a 2A peptide.

The secretory signal peptide is a peptide that transmits signals for protein secretion, the sequence of the signal peptide varies depending on the type of secreted protein, and the sequence thereof is not limited as long as the secretory signal peptide transmits signals for protein secretion.

The secretory signal peptide may be located at the N-terminus or C-terminus of a gene encoding a target protein, and is preferably located at the N-terminus and thus serves to direct the secretion of the target protein.

There may be differences in secretion efficiency depending on the secretory signal peptide, and as secretory signal peptides, it is possible to use those such as, for example, albumin, transferrin, vitellogenin, apolipoprotein B, apovitellogenin, and lysozyme. Since albumin is a representative protein that is produced and secreted in/from the liver and has high secretion efficiency, SEQ ID NO: 1, which is the sequence of albumin, may be used as the secretory signal peptide, but the secretory signal peptide is not limited thereto.

When the vector of the present invention further includes a gene encoding a secretory signal peptide downstream of the gene encoding a 2A peptide, the target protein located downstream of the secretory signal peptide may be allowed to be secreted. Specifically, when human immunoglobulin G (hIgG) Fc including a secretory signal peptide is expressed in a hepatocyte-specific manner, the human immunoglobulin G (hIgG) Fc is allowed to be secreted outside the hepatocytes.

The vector of the present invention may further include a hepatocyte-specific expressed gene-targeting single guide RNA (sgRNA).

As an RNA sequence including 20 guide sequences complementary to a target base sequence, the targeting sgRNA is for introducing a foreign gene into a specific position on a genome, and thus consists of a base sequence complementary to a base sequence of the specific position where the foreign gene on the genome is to be introduced. The hepatocyte-specific expressed gene-targeting sgRNA is a single guide RNA targeting the last intron of the hepatocyte-specific expressed gene, that is, the base sequence of the intron before the last exon and may be used to specify a position where the vector of the present invention is to be introduced, and the target base sequence should be included in the base sequence of the last intron region, and the position of the base sequence is not limited.

As a specific example, when human albumin is used as a hepatocyte-specific expressed gene, the gene-targeting single guide RNA (sgRNA) may be a single guide RNA targeting the base sequence of intron 13 of the ALB gene, and the sgRNA may consist of the base sequence of SEQ ID NO: 2, but is not limited thereto.

In addition, the present invention may provide a composition for producing a target protein.

The composition for producing a target protein includes the above-described vector, and the target protein is as described above.

The composition for producing a target protein of the present invention may be introduced into birds and used to produce the target protein. Specifically, avian germ cells are treated with the composition to transduce the avian germ cells with the vector, the germ cells may be transplanted into avian embryos to obtain a bird, and the corresponding bird may produce the target protein.

Specifically, among the target proteins, hIgG is produced in order to produce IgG, which is used as a therapeutic agent for autoimmune diseases, and currently, although there is an attempt to produce the same by an animal cell culture method, there is a limitation in preparing the same because there is a cost problem due to high concentration administration and non-human glycans produced by animal cells may cause allergic reactions. Furthermore, in the case of Fc produced by animal cell culture, α-2,6 sialylation efficiency is extremely low and fucosylation is performed, so it is difficult to expect anti-inflammatory effects due to α-2,6 sialylation and defucosylation. Therefore, human immunoglobulin G (hIgG) may be prepared using a bird that does not produce a non-human glycan.

Further, the present invention may provide a bird that expresses a gene encoding a target protein in hepatocytes. The bird of the present invention expresses a gene encoding a target protein in hepatocytes and may specifically express a gene encoding human immunoglobulin G (hIgG) Fc, and in this case, hIgG is secreted from the liver. Moreover, hIgG secreted from the liver has an advantage in that the hIgG is accumulated in egg yolk and thus can be easily extracted in addition to an advantage in that anti-inflammatory effects are improved due to a high α-2,6 sialylation rate and non-fucosylated immunoglobulins.

In the bird of the present invention, a gene encoding a target protein is introduced to express the gene in hepatocytes, the gene can be introduced by a method known in the art and expressed in hepatocytes, and for example, the gene may be introduced together with a hepatocyte-specific expressed gene. As a specific example, the bird of the present invention may be a bird (germline chimeric bird) obtained by transducing avian germ cells with the above-described vector and transplanting the transduced avian germ cells into avian embryos, and may be a homozygous progeny obtained by recrossing a heterozygous progeny obtained by crossing the bird with a wild-type bird. Homozygous expression of the target protein has the advantage of being able to produce a larger amount of protein than the heterozygous progeny generation.

Although the heterozygous progeny generation produces the target protein having both the characteristics of the germline chimeric bird and the wild-type bird, the amount of production is half that of the homozygous progeny generation. Therefore, birds produced by including the step of crossing a heterozygous progeny generation to obtain a homozygous progeny generation may be more suitable for producing a large amount of protein.

The bird of the present invention means a transgenic bird into which a gene that allows the target protein to be expressed in hepatocytes is introduced, and there are no limitations on the type of bird, as long as the bird has the characteristic of accumulating antibodies in egg yolk without producing non-human glycans. The bird may be, for example, a chicken, a quail, a pheasant, a turkey, a duck, and the like, may preferably be a chicken or a quail from the viewpoint that human immunoglobulin can be accumulated in egg yolk by the Fc region, and may preferably be a chicken from the viewpoint that proteins can be mass-produced by achieving improvements in breeding so as to enable the mass production of eggs.

The type of avian germ cell is not limited as long as the avian germ cell is suitable for the production of transgenic animals by germ cell development physiology and genomic control, and for example, the avian germ cells may preferably be primordial germ cells (PGCs) because the primordial germ cells can be cultured ex vivo for a long period of time and thus can efficiently produce germline chimeras.

In addition, the present invention may provide a method for producing a bird that expresses a gene encoding a target protein in hepatocytes.

The bird is as described above, and the method for producing a bird may include: transducing avian germ cells with the above-described vector; and obtaining a germline chimeric bird by transplanting the transduced germ cells into avian embryos.

The type of avian germ cell is not limited as long as the avian germ cell is suitable for the production of transgenic animals by germ cell development physiology and genomic control, and for example, the avian germ cells may preferably be primordial germ cells (PGCs) because the primordial germ cells can be cultured ex vivo for a long period of time and thus can efficiently produce germline chimeras.

The transduced germ cells may be implanted into avian embryos to produce a germline chimeric bird, and for example, may be transplanted into the aorta of avian embryos.

The method for producing a bird may further include: obtaining a heterozygous progeny generation by crossing the germline chimeric bird with a wild-type bird; and obtaining a homozygous progeny generation by crossing the heterozygous progeny generation.

Although the heterozygous progeny generation produces a protein encoded by a foreign gene having both the characteristics of the germline chimeric bird and the wild-type bird, the amount of production is half that of the homozygous progeny generation. Therefore, birds produced by including the step of crossing a heterozygous progeny generation to obtain a homozygous progeny generation may be more suitable for producing a large amount of protein.

Furthermore, the present invention may provide a method for producing a target protein.

The method for producing a target protein may include purifying a target protein in the egg yolk or blood of the bird of the present invention.

The target protein may be purified by a general protein purification method known in the art, and may be specifically purified by chromatography, and the chromatography may be, for example, ion exchange chromatography, size exclusion chromatography, and affinity chromatography, but is not limited thereto.

Further, the present invention may provide a target protein obtained from the egg yolk or blood of a bird expressing a gene encoding the target protein in hepatocytes.

The target protein may be human immunoglobulin G (hIgG), factor VIII, factor IX, alpha-1-antitrypsin, erythropoietin, growth hormone, colony-stimulating factor, interferon, insulin and glucagon-like peptide-1, a CD20 antibody, a HER2 antibody, an EGFR antibody or a TNF-alpha antibody, and may be obtained in egg yolk or blood because the target protein is produced and secreted in/from the liver by a hepatocyte-specific expressed gene and a secretory signal peptide and accumulated in egg yolk, and more specifically, the target protein may be a therapeutic agent for autoimmune diseases when the target protein is human immunoglobulin G (hIgG).

Hereinafter, the present invention will be described in detail through Examples. However, the following Examples are only for exemplifying the present invention, and the content of the present invention is not limited by the following Examples.

### Examples

### 1. Experimental Method

### (1) Construction of CRISPR/Cas9 Expression Plasmid

An all-in-one CRISPR/Cas9 plasmid targeting intron 13 of an ALB gene was constructed. The CRISPR kit used for the construction of a CRISPR/Cas9 plasmid (SEQ ID NO: 3) was provided by Takashi Yamamoto (Addgene, #1000000054) and the backbone CRISPR/Cas9 plasmid was provided by Feng Zhang (Addgene, #62988). Sense and antisense oligonucleotides were designed and synthesized at Bionics in order to insert a guide RNA (gRNA) sequence into the backbone CRISPR/Cas9 plasmid. The sense and antisense oligonucleotides were annealed under thermocycling conditions of 30 seconds at 95° C., 2 minutes at 72° C., 2 minutes at 37° C., and 2 minutes at 25° C.

### (2) Construction of Donor Plasmid

To insert a target gene into intron 13 of chicken ALB, a donor plasmid (SEQ ID NO: 4) including the intron 13 of ALB, the exon 14 of ALB without a stop codon, a T2A coding sequence, an ALB signal peptide coding sequence, a hIgG Fc coding sequence, an ALB 3′UTR and a BGH poly A site was synthesized at BIONEER. Thereafter, a puromycin resistance gene with a cytomegalovirus promoter was cloned and inserted into the synthesized donor plasmid. The donor plasmid has an sgRNA recognition site of intron 13 of ALB for linearization.

### (3) Culture of Chicken Primordial Germ Cells (PGCs)

White leghorn (WL) PGCs were maintained and sub-cultured in knock-out DMEM supplemented with 20% PBS (Thermo Fisher Scientific), 2% chicken serum (Thermo Fisher Scientific), 1× nucleosides (MilliporeSigma), 2 mML-glutamine, 1× non-essential amino acids, beta-mercaptoethanol, 10 mM sodium pyruvate, 1× antibiotic-antimycotic (Thermo Fisher Scientific) and a human basic fibroblast growth factor (10 ng/ml; Komabiotech). Chicken PGCs were cultured in an incubator at 37° C. under an atmosphere of 5% CO₂ and 60 to 70% relative humidity. The PGCs were sub-cultured on mitomycin-inactivated mouse embryonic fibroblasts at 5-6 day intervals via gentle pipetting.

### (4) Transduction of PGCs and Puromycin Selection for Insertion of Target Gene

For genome editing in chicken PGCs, donor plasmids (4 μg) and CRISPR/Cas9 plasmids (4 μg) were co-introduced into PGCs (1×10⁵ cells) cultured with 8 μl of Lipofectamine 2000 reagent suspended in 1 ml of Opti-MEM. 4 hours after transduction, the transduction mixture was replaced with a PGC culture medium. Puromycin (1 μg/ml) was added to the culture medium 1 day after transduction and the selection period was 2 days.

### (5) Production of ALB-hIgG Fc-Tagged Genome-Edited Chickens

To produce ALB-hIgG Fc-tagged genome-edited chickens, a window was cut at the sharp end of the Korean Ogye recipient egg, and 3000 or more ALB-hIgG Fc-tagged genome-edited WL PGCs were transplanted into the dorsal aorta of Hamburger and Hamilton (HH) stage 14-17 recipient embryos. The egg window was sealed with paraffin film, and the eggs were incubated with the pointed end down until hatching. After sexual maturation, sperm from the male recipient chickens was evaluated by breed-specific PCR, and the chickens that have WL sperm were mated with WT female chickens. Germline-chimeric chickens were identified based on offspring feather color and subsequent genomic DNA analysis.

### (6) Detection of ALB-hIgG FC Tag

To identify modified alleles in chicken PGCS and ALB-hIgG FC tagged genome-edited chickens, genomic DNA was analyzed using knock-in PCR analysis as a specific primer for intron 13 of ALB and the hIG Fc coding sequence. All reactions were performed under the same conditions, with a total PCR volume of 20 μl including 100 ng of genomic DNA, 10×PCR buffer, 0.4 μl dNTPs (10 mM each), μM of each primer, and 0.5 U Taq polymerase (Bionics) under the following thermocycling conditions: 5 minutes at 95° C., 30 seconds at 95° C., 30 seconds at 60° C., 30 seconds at 72° C., and finally, 5 minutes at 72° C. For sequencing analysis, amplicons were annealed into the pGEM-T easy vector and sequenced using an ABI Prism 3730 XL DNA Analyzer. The sequences were compared with assembled genomes using BLAST.

### (7) Western Blot Analysis of ALB-hIgG Fc Tagged Genome-Edited Chicken Serum

For western blotting, ALB-hIgG Fc tagged genome-edited chicken serum was collected, diluted 10-fold in DW and mixed with the same volume of 2× Laemmli sample buffer. In order to obtain unambiguous detection, serum proteins were separated on a 15% SDS-polyacrylamide gel. Degraded proteins were transferred to a Hybond 0.45 μm polyvinylidene difluoride (PVDF) membrane (GE Healthcare Life Sciences) and blocked in 5% BSA at room temperature for 1 hour. The membrane was incubated with a goat anti-human IgG primary antibody (Alpha Diagnostics) diluted in a blocking buffer (1:1000) overnight and then with an HRP-conjugated secondary antibody diluted 1:5000 for 1 hour. Immunoreactive proteins were visualized with an ECL Western blot detection system (GE Healthcare Life Sciences). Wild-type WL chicken serum was used as a control.

### (8) SDS-PAGE and Coomassie Blue Staining

For Coomassie blue staining, ALB-hIgG Fc tagged genome-edited chicken serum was collected, diluted 5-, 25- and 50-fold in DW and mixed with the same volume of 2× Laemmli sample buffer. Serum proteins were separated on a 15% SDS-polyacrylamide gel. After separation, the gel was stained with a Coomassie blue staining solution at room temperature for 2 hours and desalted overnight using a desalting buffer.

### (9) Enzyme-Linked Immunosorbent Assay (ELISA)

The concentration of hIgG Fc was determined using a human IgG enzyme-linked immunosorbent assay (ELISA) kit (abeam #195215) according to the manufacturer's instructions. This kit uses the double antibody sandwich method, and the optical density is proportional to the amount of monoclonal antibody present. The concentration of hIgG Fc was determined relative to the optical density of a standard protein.

### (10) Purification of hIgG Fc in Serum

For human IgG purification, 4 M ammonium sulfate was slowly added to chicken serum, and the mixture was stirred at 4° C. overnight and then centrifuged at 1000 g at 4° C. for 30 minutes. The lysate was resuspended in the same volume of 1×PBS as the original serum volume. The mixture was then dialyzed against 20 mM sodium phosphate buffer (pH 7.1). The sample was loaded onto a protein A column (GE Healthcare Bio Science) and proteins were eluted with a 100 ml gradient of 100 mM citric acid (pH 2.8). The proteins were additionally purified and fractionated by size exclusion chromatography (SEC) using a HiLoad Superdex 75 column (GE-Healthcare Bio Science) pre-equilibrated with 20 mM Tris-HCl and 175 mM NaCl (pH 7.4).

### (11) N-Glycosylation Pattern Analysis of hIgG Fc

N-glycans of hIgG Fc were analyzed by UPLC/MS. Briefly, purified hIgG Fc was incubated with 10 mM dithiothreitol in 50 mM ammonium bicarbonate buffer at 56° C. for 30 minutes. PNGase F (500 units) was added thereto, and the resulting mixture was incubated at 37° C. for 16 hours. After 2 mL of cold ethanol was added to the reaction sample in order to precipitate the deglycosylated Fc, the resulting mixture was incubated at 20° C. for 4 hours. After incubation, samples were centrifuged at 10,000 g for 10 minutes, and the supernatant, from which N-glycans had been released, was transferred to a new tube and completely dried using a Speed-Vac concentrator. The dried samples were further labeled with procainamide for fluorescence analysis. 350 mL of dimethyl sulfoxide and 150 mL of glacial acetic acid were added to glass vials for labeling. Subsequently, 13 mg of procainamide was added to the mixture (100 mL) and the solution was completely dissolved. The mixture was completely dissolved after adding 6 mg of sodium cyanoborohydride (NaBH₃CN). 5 μl of the mixture was added to the completely dried N-glycan samples and allowed to react at 37° C. for 16 hours. In order to remove excess procainamide reagent, solid phase extraction was performed using an S-cartridge. The S-cartridge was activated and equilibrated by mixing 1 mL of HPLC grade H20, 1 mL of 30% acetic acid (5 times) and 1 mL of 100% acetonitrile (4 times). The procainamide-labeled sample was mixed with 100 mL of 100% acetonitrile, the resulting mixture was loaded onto an S-cartridge, and washed with an excess of procainamide reagent (5 times) instead of a portion of the fluorescent label in 1 mL of acetonitrile. Next, 1.5 mL of H2O was added to elute the procainamide-labeled N-glycans. Procainamide-labeled N-glycan samples were analyzed and quantified by UPLC/FLD coupled with mass spectrometry. An ACQUITY UPLC BEH glycan column (Waters iClass UPLC) with a fluorescence detector was used for separation and detection of N-glycans. LC conditions are as follows: flow rate (0.5 mL/min), column temperature (60° C.), mobile phase buffer A (100 mM ammonium formate, pH 4.5), buffer B (100% acetonitrile), injection volume (8 mL), linear gradient (75-60% B for 46.5 minutes, 60-0% B for 1.5 minutes, 0% B for 1 minute, 0-75% B for 1 minute, and 75% B for 13 minutes). A high resolution mass spectrometry, Triple-TOP MS (AB SCIEX, Concord, Ontario, Canada) was used for N-glycan identification. The N-glycan distribution was analyzed with Empower (Waters).

### 2. Experimental Results

### (1) Production of ALB-hIgG Tagged Genome-Edited Chickens

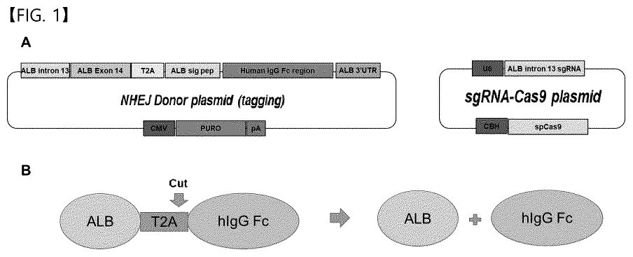

In order to produce gene-edited chickens that produce hIgG Fc with enhanced anti-inflammatory efficacy, the present invention was intended to produce hIgG Fc in the liver using the endogenous promoter of a liver-specifically expressed albumin (ALB) gene. For this purpose, it was designed such that hIgG Fc would be linked to the ALB protein followed by a 2A peptide for translation, and then ALB and hIgG Fc would be separated from the cytoplasm of hepatocytes by the 2A peptide. In this case, it was designed such that hIgG Fc separated from ALB could be normally secreted into the blood from hepatocytes by inserting the ALB signal peptide sequence before the sequence of hIgG Fc (FIGS. 1A and 1B). For this purpose, a donor plasmid and a CRISPR/Cas9 plasmid were constructed, and through this, ALB-hIgG Fc tagged genome-edited chickens were produced (FIGS. 1C and 1D).

### (2) Confirmation of hIgG Production in Hepatocytes Of Genome-Edited Chickens

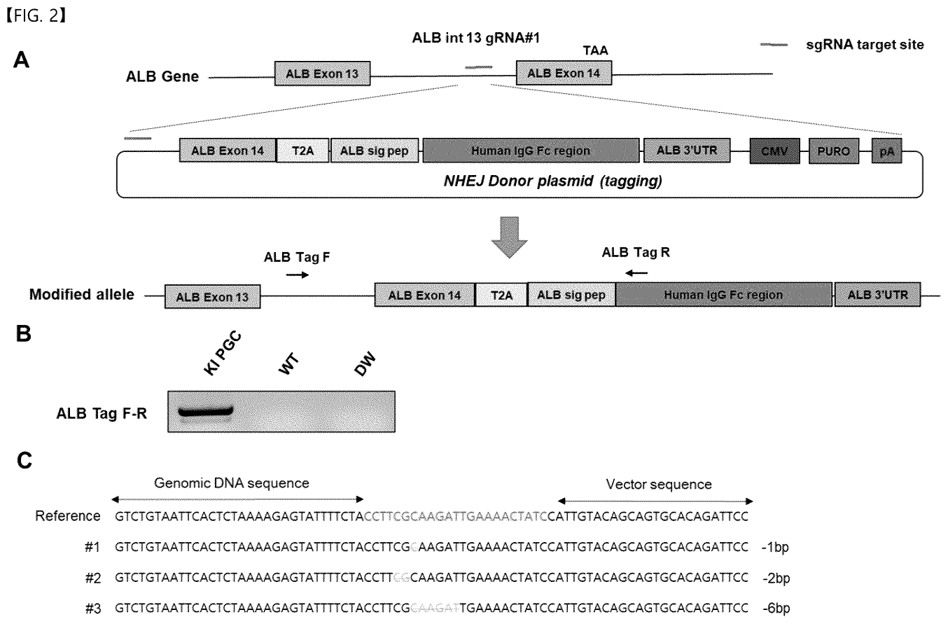

In order to confirm whether hIgG was normally secreted from hepatocytes of the produced genome-edited chicken and present in the blood, western blot was performed by collecting blood. As a result, it was confirmed that hIgG Fc was normally produced and circulated in the blood of ALB-hIgG tagged genome-edited chickens. In addition, it was confirmed that there was also a form in which ALB and Fc were not separated because some 2A peptides were not completely processed (FIG. 2A). In addition, it was confirmed that hIgG Fc was present in the blood of ALB-hIgG tagged genome-edited chickens through Coomassie blue staining and ELISA (FIGS. 2B and 2C). From this, it can be seen that hIgG Fc is normally produced in chicken hepatocytes by ALB tagging and continuously secreted into the blood.

### (3) N-Glycosylation Pattern Analysis of Produced hIgG Fc

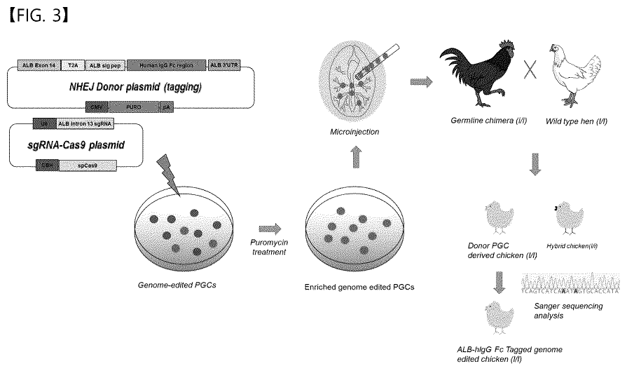

Next, in order to analyze the N-glycosylation pattern of hIgG Fc produced in the chicken liver, hIgG Fc was purified from blood by Protein A affinity chromatography and size exclusion chromatography, and the N-glycosylation pattern was analyzed through UPLC/MS after isolating the N-glycans of the purified hIgG Fc. As a result of the analysis, it was confirmed that approximately 30% of the hIgG Fc produced in the chicken liver was sialylated Fc, and that no fucosylation was performed (FIGS. 3A and 3B).

1. A vector into which a hepatocyte-specific expressed gene, a gene encoding an Fc region of an antibody, and a gene encoding a target protein are introduced.

2. The vector of claim 1, wherein the hepatocyte-specific expressed gene encodes at least one selected from the group consisting of albumin, beta-livetin, vitellogenin, apovitellenin, alpha-2-macroglobulin, apolipoprotein, transferrin and fibrinogen.

3. The vector of claim 1, wherein the hepatocyte-specific expressed gene encodes albumin.

4. The vector of claim 1, wherein the vector is inserted into ALB 13 intron.

5. The vector of claim 1, wherein the target protein is at least one selected from the group consisting of human immunoglobulin G (hIgG), factor VIII, factor IX, alpha-1-antitrypsin, erythropoietin, growth hormone, colony-stimulating factor, interferon, insulin and glucagon-like peptide-1, a CD20 antibody, a HER2 antibody, an EGFR antibody and a TNF-alpha antibody.

6. The vector of claim 1, wherein the hepatocyte-specific expressed gene does not include a stop codon thereof.

7. The vector of claim 1, further comprising a gene encoding a 2A peptide between the hepatocyte-specific expressed gene and a gene encoding a target protein.

8. The vector of claim 7, wherein the 2A peptide consists of 18 to 22 amino acid sequences.

9. The vector of claim 7, wherein the 2A peptide is at least one selected from the group consisting of T2A, P2A, F2A and E2A.

10. The vector of claim 7, further comprising a gene encoding a secretory signal peptide downstream of the gene encoding a 2A peptide.

11. The vector of claim 10, wherein the vector is configured in the order the hepatocyte-specific expressed gene-the gene encoding a 2A peptide-the gene encoding a secretory signal peptide-the gene encoding an Fc region of an antibody-the gene encoding a target protein.

12. The vector of claim 10, wherein the secretory signal peptide is a amino acid sequences set forth SEQ ID NO: 1.

13. A method for producing a bird, the method comprising: transducing avian germ cells with the vector of claim 1; and obtaining a germline chimeric bird by transplanting the transduced germ cells into avian embryos.

14. The method of claim 13, further comprising: obtaining a heterozygous progeny generation by crossing the germ line chimeric bird with a wild-type bird; and obtaining a homozygous progeny generation by crossing the heterozygous progeny generation.

15. The method of claim 13, wherein the bird is a chicken, a quail, a pheasant, a turkey, or a duck.

16. The method of claim 13, wherein the avian germ cells are primordial germ cells (PGCs).

17. A method for producing a target protein, the method comprising purifying a target protein in an egg yolk or blood of a bird transduced by the vector of claim 1.

18. The method of claim 17, wherein the bird is co-transduced with the vector of claim 1 and a vector into which CRISPR/Cas system is introduced.

19. The method of claim 18, wherein the CRISPR/Cas system includes a guide RNA targets the hepatocyte-specific expressed gene.

20. The method of claim 17, wherein the purifying a target protein is performed by a chromatography.
